(19) United States
(12) Patent Application Publication
(10) Pub. No.: US 2021/0181844 A1
(43) Pub. Date: Jun. 17, 2021

(54) USER INTERFACE SYSTEM AND AN OPERATION METHOD THEREOF

(71) Applicants: HYUNDAI MOTOR COMPANY, Seoul (KR); KIA MOTORS CORPORATION, Seoul (KR); Research & Business Foundation SUNGKYUNKWAN UNIVERSITY, Suwon-si (KR)

(72) Inventors: Soon Kwon Paik, Yongin-si (KR); Tae Il Kim, Pyeongtaek-si (KR); Joo Hwan Shin, Suwon-si (KR)

(73) Assignees: HYUNDAI MOTOR COMPANY, Seoul (KR); KIA MOTORS CORPORATION, Seoul (KR); Research & Business Foundation SUNGKYUNKWAN UNIVERSITY, Suwon-si (KR)

(21) Appl. No.: 16/940,150

(22) Filed: Jul. 27, 2020

(30) Foreign Application Priority Data
Dec. 16, 2019 (KR) 10-2019-0167673

(51) Int. Cl. G06F 3/01; G06F 3/0484

(52) U.S. Cl. CPC G06F 3/015; A61B 5/0482; G06F 3/0484

(57) ABSTRACT

A user interface system and an operation method therefor are disclosed. The user interface system includes an electroencephalogram (EEG) detection device that detects event-related potential information by measuring an EEG of a user and includes a user input recognition device that recognizes and corrects an input recognition error by analyzing the event-related potential information when the user's input is recognized.

### CROSS-REFERENCE TO RELATED APPLICATION

This application claims the benefit of priority to Korean Patent Application No. 10-2019-0167673, filed in the Korean Intellectual Property Office on Dec. 16, 2019, the entire contents of which are incorporated herein by reference.

### TECHNICAL FIELD

The present disclosure relates to a user interface system and an operation method therefor.

### BACKGROUND

Speech recognition technology is growing in importance in the automotive field. Speech recognition technology may control a vehicle using speech without any physical manipulation of a driver, thus solving risks that may be caused by, for example, manipulation of navigation or convenience functions while driving. Accordingly, speech recognition technology is used in various platforms such as an artificial intelligence virtual assistant service and a vehicle control service.

Such conventional speech recognition technology is difficult to recognize speech without errors due to different pronunciations and intonations of users. Accordingly, studies have been actively conducted to reduce recognition errors upon speech recognition (i.e., user input recognition).

### SUMMARY

The present disclosure has been made to solve the above-mentioned problems occurring in the prior art while advantages achieved by the prior art are maintained intact.

An aspect of the present disclosure provides a user interface system and an operation method. The system and method analyze an event-related potential pattern by measuring a user's electroencephalogram (EEG) and recognize and correct a user input recognition error based on an analysis result.

The technical problems to be solved by the present inventive concept are not limited to the aforementioned problems. Any other technical problems not mentioned herein should be clearly understood from the following description by those having ordinary skill in the art to which the present disclosure pertains.

According to an aspect of the present disclosure, a user interface system includes an EEG detection device that detects event-related potential information by measuring an EEG of a user. The user interface system also includes a user input recognition device that recognizes and corrects an input recognition error by analyzing the event-related potential information when an input of the user is recognized.

The event-related potential may be a potential change occurring in a brain of the user with respect to a feedback output from the user input recognition device.

The EEG detection device may include: an electrode attached to a scalp of the user to receive an EEG signal; a first amplifier that primarily amplifies the EEG signal; a noise filter that removes noise from the EEG signal primarily amplified by the first amplifier; a second amplifier that secondly amplifies the EEG signal from which the noise has been removed; a controller that extracts the event-related potential information from the EEG signal secondly amplified by the second amplifier; and a first communication device that transmits the event-related potential information to the user input recognition device using wireless communication according to an instruction of the controller.

The electrode may be made of an ultra-thin film of 1pm or less.

The noise filter may be implemented with a Driven Right Leg Circuit (DRLC) including two OP-AMPs and resistors.

The user input recognition device may include: a second communication unit that receives the event-related potential information transmitted from the EEG detection device through wireless communication; an input recognition device that recognizes the input of the user; and a processor that outputs a feedback according to a recognition result of the input of the user of the input recognition device and recognizes and corrects an input recognition error based on the event-related potential information.

The processor may determine the input recognition error when a P300 potential component in the event-related potential information is found.

The P300 potential component may be a peak appearing around 300 msec after output of the feedback.

The processor may correct the input recognition error by requesting the user to re-input data or identifying an intention of the user through a query.

According to an aspect of the present disclosure, a method of operating a user interface system includes: detecting event-related potential information through measurement of an EEG of a user when recognizing an input of the user; recognizing an input recognition error by analyzing the event-related potential information; and correcting the input recognition error.

The event-related potential may be a potential change occurring in a brain of the user with respect to a feedback output from a user input recognition device.

The detecting of the event-related potential information may include: recognizing the input of the user; outputting a feedback according to a result of recognizing the input; measuring an EEG signal of the user upon output of the feedback; and extracting the event-related potential information from the EEG signal.

The measuring of the EEG signal of the user may include primarily amplifying the EEG signal, removing noise included in the primarily-amplified EEG signal, and secondly amplifying the EEG signal from which the noise has been removed.

The recognizing of the input recognition error may include determining that an input recognition error occurs when a P300 potential component is found in the event-related potential information.

The P300 potential component may be a peak appearing around 300 msec after output of the feedback.

The correcting of the input recognition error may include correcting the input recognition error by requesting the user to re-input data or identifying an intention of the user through a query.

### BRIEF DESCRIPTION OF THE DRAWINGS

The above and other objects, features and advantages of the present disclosure will be more apparent from the following detailed description taken in conjunction with the accompanying drawings:

FIG. 1 is a block diagram illustrating a user interface system according to an embodiment of the present disclosure;

FIGS. 2-4 are diagrams for describing an event-related potential;

FIG. 5 is a block diagram illustrating an electroencephalogram (EEG) detection device shown in FIG. 1;

FIG. 6 is a view showing an ultra-thin electrode according to the present disclosure;

FIG. 7 is a view of a PCB design of an EEG detection device according to the present disclosure;

FIGS. 8 and 9 are application examples of an EEG detection device according to an embodiment of the present disclosure; and

FIG. 10 is a flowchart illustrating a method of operating a user interface system according to an embodiment of the present disclosure.

### DETAILED DESCRIPTION

Hereinafter, some embodiments of the present disclosure are described in detail with reference to the drawings. In adding the reference numerals to the components of each drawing, it should be noted that identical or equivalent components are designated by identical numerals even when they are displayed on other drawings. Further, in describing the embodiment of the present disclosure, a detailed description of well-known features or functions has been omitted in order not to unnecessarily obscure the gist of the present disclosure.

In describing the components of the embodiment according to the present disclosure, terms such as first, second, “A”, “B”, (a), (b), and the like may be used. These terms are merely intended to distinguish one component from another component. The terms do not limit the nature, sequence, or order of the constituent components. Unless otherwise defined, all terms used herein, including technical or scientific terms, have the same meanings as those generally understood by those having ordinary skill in the art to which the present disclosure pertains. Such terms as those defined in a generally used dictionary are to be interpreted as having meanings equal to the contextual meanings in the relevant field of art. Such terms are not to be interpreted as having ideal or excessively formal meanings unless clearly defined as having such in the present application.

The present disclosure relates to a technique for recognizing and correcting a recognition error for a user input through event-related potential (EEG) analysis. The event-related potential may refer to the electrical activity of a brain after a particular stimulus is provided, and may consist of several peaks or components representing positive and negative potentials. P300, which is a peak appearing around 300 msec with a positive potential among event-related potentials, may be related to a recognition process and an information processing process.

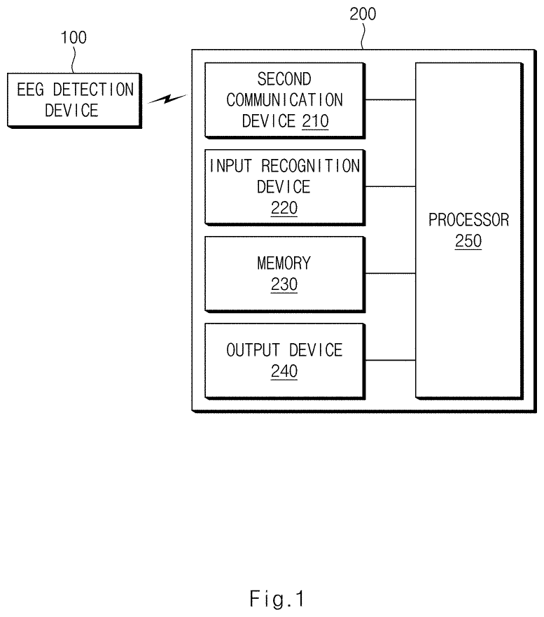

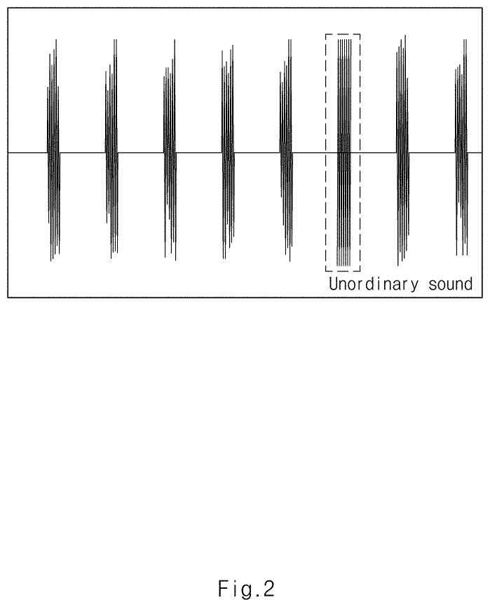

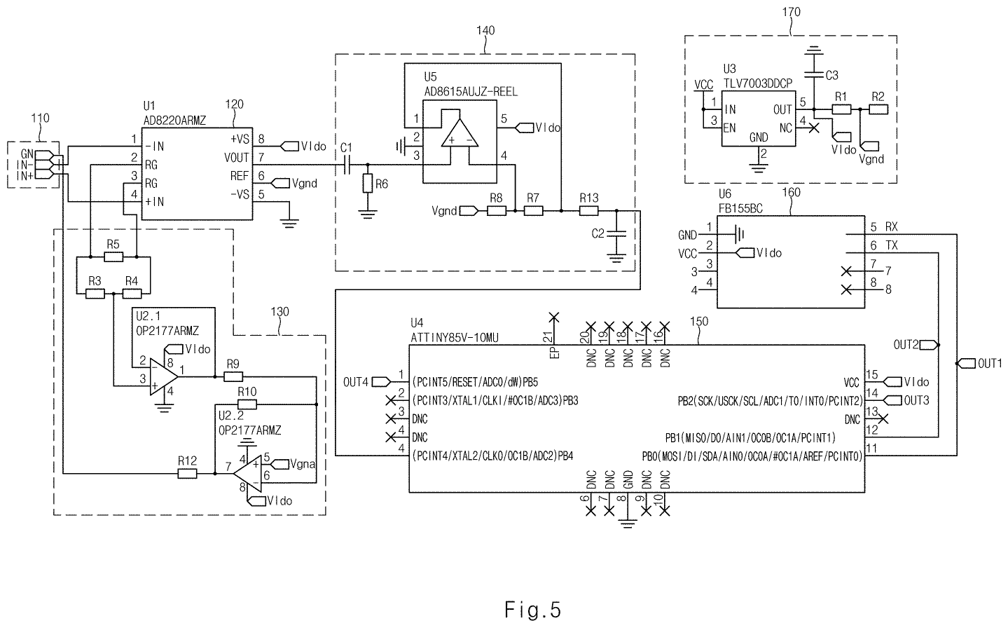

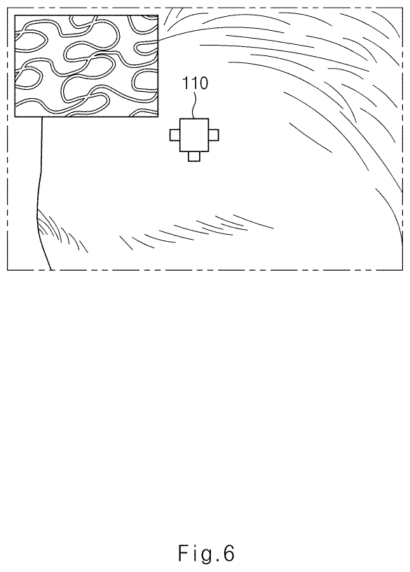

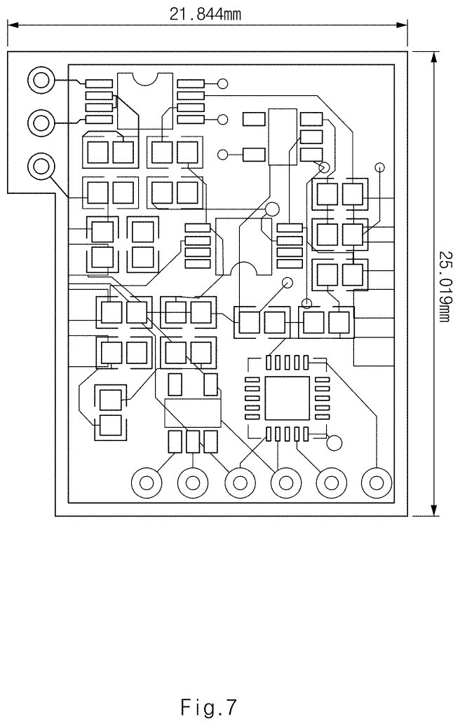

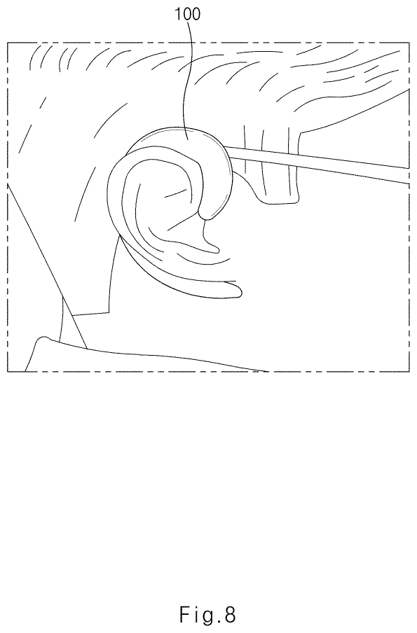

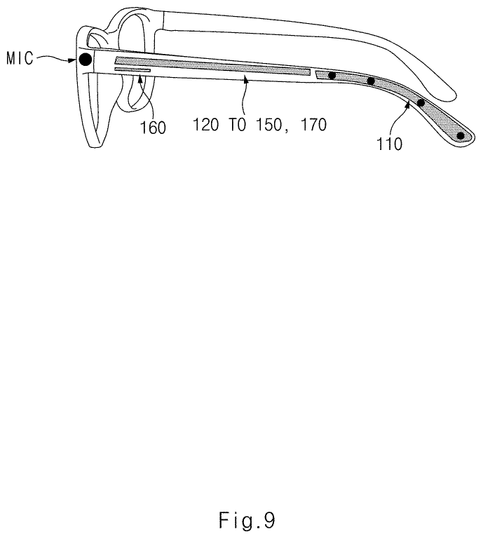

FIG. 1 is a block diagram illustrating a user interface system according to an embodiment of the present disclosure. FIGS. 2-4 are diagrams for describing an event-related potential. FIG. 5 is a block diagram illustrating an electroencephalogram (EEG) detection device shown in FIG. 1. FIG. 6 is a view showing an ultra-thin electrode according to the present disclosure. FIG. 7 is a view of a PCB design of an EEG detection device according to the present disclosure. FIGS. 8 and 9 are application examples of an EEG detection device according to an embodiment of the present disclosure.

Referring to FIG. 1, a user interface system may include an EEG detection device 100 and a user input recognition device 200, which exchange data with each other in real time through wireless communication. In the present embodiment, a case in which the user input recognition device 200 recognizes a user's speech is described as an example to help understand the present disclosure. However, the present disclosure is not limited thereto, and the user's touch input or button input may be recognized.

The EEG detection device 100 may be an EEG sensor that measures (detects) an EEG signal of a user (e.g., a driver). The EEG detection device 100 may extract an event-related potential (ERP) from the EEG signal and provide the event-related potential to the user input recognition device 200. The event-related potential may be one of multiple potentials detectable from the EEG and may be closely related to a decision process for user's decision making and stimulus. The event-related potential may be an endogenous potential that occurs according to an individual's response and decision to each stimulus, regardless of the physical characteristics of the stimulus. Typically, a P300 potential component in an event-related potential may be a potential component that is caused when an abnormal stimulus is intermittently involved with natural, problem-free, and normal visual, auditory, and/or tactile stimuli, which are expected to be judged by the user. The P300 potential component may be the maximum peak that appears approximately 300 msec after the time point at which the abnormal stimulus is involved.

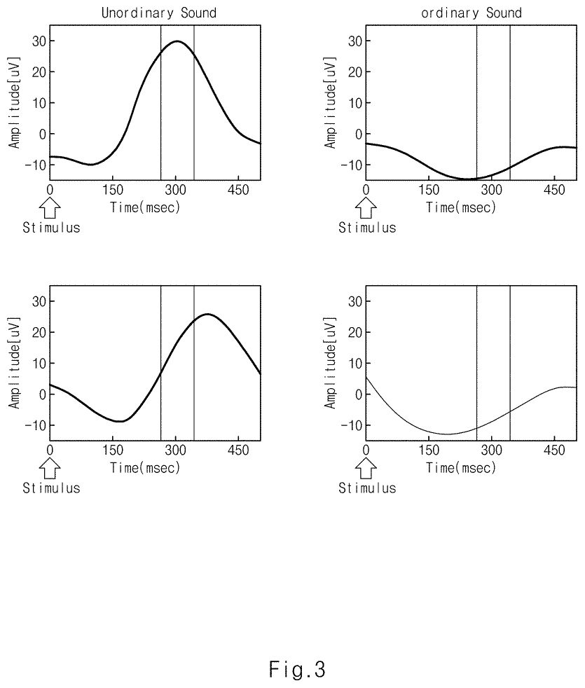

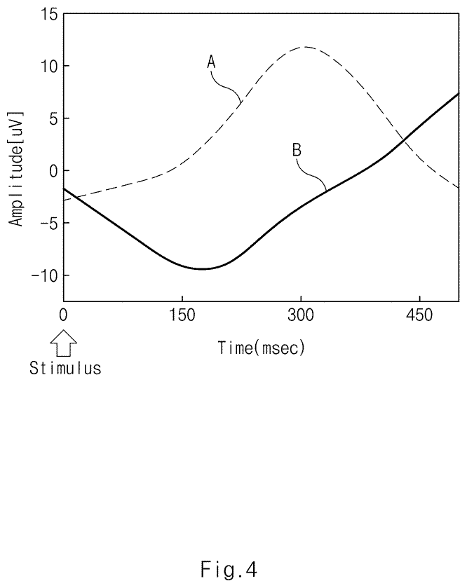

For example, when a speech file, in which an unordinary sound is inserted as a stimulus signal between repeated sounds as shown in FIG. 2, is played, an event-related potential does not occur in an EEG waveform for an ordinary sound as shown in FIG. 3. However, an event-related potential occurs in an EEG waveform for a specific sound inserted as a stimulus signal. In particular, referring to FIG. 4, when the stimulus signal is generated (A) and when the stimulus signal is not generated (B), a potential difference is large occurring at around 300 msec.

As illustrated in FIG. 5, the EEG detection device 100 may include an electrode 110, a first amplifier 120, a noise filter 130, a second amplifier 140, a controller 150, a first communication device 160, and a power supply 170.

The electrode 110 may be attached to the user's scalp, forehead, or the back of the user's ear to receive an EEG signal. As shown in FIG. 6, the electrode 110 may be made of an ultra thin film of about 1 pm. The electrode 110 may be fabricated to have a desired shape and design through patterning on a silicon substrate, and transferred to a very thin tattoo paper through a transfer process. The electrode 110 is in close contact with a curved skin surface to minimize the impedance between the electrode 110 and the skin. The electrode 110 is thereby resistant to noise caused by the user's movement.

The first amplifier 120 is an instrumentation amplifier and may primarily amplify an EEG signal input through the electrode 110. The first amplifier 120 may amplify the EEG signal at a predetermined ratio.

The noise filter 130 may remove (filter out) noise from the EEG signal primarily amplified by the first amplifier 120. The noise filter 130 may be implemented with a DRLC (Driven Right Leg Circuit). The noise filter 130 may include two operational amplifiers, i.e., two OP-AMPs and resistors.

The second amplifier 140 may secondly amplify the EEG signal from which the noise is removed. The second amplifier 140 may amplify the EEG signal from which the noise is removed at a predetermined ratio.

The controller 150 may control the overall operation of the EEG detection device 100. The controller 150 may be implemented with at least one of an application specific integrated circuit (ASIC), a digital signal processor (DSP), a programmable logic device (PLD), field programmable gate array (FPGAs), a central processing unit (CPU), microcontrollers, and microprocessors.

The controller 150 may extract event-related potential information from the EEG signal (i.e., secondly-amplified EEG signal) output from the second amplifier 140. The controller 150 may receive a user input recognition operation notification from the user input recognition device 200 through the first communication device 160. When the controller 150 receives the user input recognition operation notification, the controller 150 may measure an EEG signal through the electrode 110. In addition, when receiving feedback information transmitted from the user input recognition device 200 through the first communication device 160, the controller 150 may extract event-related potential information from the measured EEG signal.

The first communication device 160 may transmit (transfer) the extracted event-related potential according to an instruction of the controller 150. The first communication device 160 may transmit and receive data using wireless communication technology such as Bluetooth, Near Field Communication (NFC), Radio Frequency Identification (RFID), Infrared Data Association (IrDA), Ultra Wideband (UWB), ZigBee, and/or Wi-Fi.

The power supply 170 may be powered by an external power supply voltage common collector (VCC) to supply power required for the operation of each of the components 110 to 160 under the control of the controller 150. Here, the external power supply VCC may be implemented with an external battery. The power supply 170 may step-down a voltage input from the external power supply VCC to a voltage required for the operation of each of the components 110 to 160. The power supply 170 may be implemented with a low drop out regulator.

The EEG detection device 100 may be completed by designing and manufacturing a printed circuit board (PCB) as shown in FIG. 7, based on the circuit diagram shown in FIG. 5, and arranging the above components on the manufactured PCB. The EEG detection device 100 may be applied to an ear set, glasses, or the like. When the EEG detection device 100 is applied to the ear set as shown in FIG. 8, the electrode 110 may be disposed on a body surface of the ear set in contact with the skin behind the user's ear. On the other hand, when the EEG detection device 100 is applied to the glasses as shown in FIG. 9, the electrode 110 may be disposed on a contact surface of a leg of the glasses in contact with the skin behind the user's ear and the components 120 to 170 constituting the EEG detection device 100 may be disposed in the body of the leg of the glasses. In addition, the EEG detection device 100 may be implemented integrally with the user input recognition device 200, which is described below. In addition, the EEG detection device 100 may be manufactured by being applied to a bone conduction headset.

The user input recognition device 200 may be mounted in a vehicle to recognize data input by a user, i.e., a user input, and may include a second communication device 210, an input recognition device 220, a memory 230, and an output device 240 and a processor 250.

The second communication device 210 may perform wireless communication with the EEG detection device 100. Wireless communication technologies may include Bluetooth, Near Field Communication (NFC), Radio Frequency Identification (RFID), Infrared Data Association (IrDA), Ultra Wideband (UWB), ZigBee, and/or Wi-Fi and the like.

In addition, the second communication device 210 may allow the user input recognition device 200 to communicate with an electric control unit (ECU) mounted in the vehicle. The second communication device 210 may exchange data and/or control commands with an electronic control device using an In-Vehicle Network (IVN) such as a controller area network (CAN), a media oriented systems transport (MOST) network, a local interconnect network (LIN), an ethernet, and/or an X-by-Wire (Flexray).

The input recognition device 220 may acquire speech information (utterance information) of the user through at least one microphone (not shown) installed in the vehicle. The input recognition device 220 may convert a speech signal into text through signal processing when a speech signal spoken by a user (e.g., a driver and/or a passenger) in a vehicle is input. In this example, a microphone (not shown) is a sound sensor that receives an external acoustic speech signal and converts the external acoustic speech signal into an electrical signal. Various noise removal algorithms may be implemented in the microphone to remove noise, which is input along with the acoustic speech signal. In other words, the microphone may remove noise, occurring during driving or introduced from the outside, from the acoustic speech signal input from the outside and output the acoustic speech signal.

In the present embodiment, the input recognition device 220 may recognize speech data (speech information) spoken by the user using at least one or more of various known speech recognition technologies. Thus, a detailed description of the speech recognition technology has been omitted.

In addition, the input recognition device 220 may recognize a user input (user data) input through an input device such as a keyboard, a keypad, a button, a switch, a touch pad, and/or a touch screen.

The memory 230 may store a program for the operation of the processor 250, and may temporarily store input and/or output data. The memory 230 may be implemented with at least one of storage media (recording media), such as a flash memory, a hard disk, an SD card (Secure Digital Card), a random access memory (RAM), a static random access memory (SRAM), a Read Only Memory (ROM), a Programmable Read Only Memory (PROM), an Electrically Erasable and Programmable ROM (EEPROM), an Erasable and Programmable ROM (EPROM), a register, a removable disk, and a web storage.

The output device 240 may output a progress status and a result according to the operation of the processor 250 in the form of visual information and/or auditory information. The output device 240 may include a display and/or a speaker. In this example, the display may be implemented with a touch screen combined with a touch sensor, and thus may be used as an input device as well as an output device.

The output device 240 may output a result produced from the user input recognition according to an instruction of the processor 250 to feed the result back to the user.

The processor 250 may control overall operation of the user input recognition device 200. The processor 250 may be implemented with at least one of an application specific integrated circuit (ASIC), a digital signal processor (DSP), a programmable logic device (PLD), field programmable gate array (FPGAs), a central processing unit (CPU), microcontrollers, and microprocessors.

When the processor 250 recognizes the user's input (e.g., a speech signal) through the input recognition device 220, the processor 250 may output a feedback according to a result of the input recognition. For example, when the user utters “search for 00 department store”, the processor 250 may recognize the user's utterance information through the input recognition device 220 and output a speech message such as “search for OO department store” as a feedback according to a recognition result to the output device 240.

The processor 250 may transmit feedback information including whether the feedback is output and a feedback output time, or the like, to the EEG detection device 100 when the feedback is output.

Thereafter, the processor 250 may receive event-related potential information transmitted from the EEG detection device 100 through the second communication device 210. The processor 250 may analyze the received event-related potential information to determine whether an input recognition error (malfunction) is present (whether an error occurs). The processor 250 may determine whether a P300 potential component (P300 pattern) exists in the event-related potential information. In other words, the processor 250 may determine whether a P300 pattern is found in the EEG of the user.

When the P300 potential component exists in the event-related potential information, the processor 250 may determine that the input recognition error is present. When the processor 250 recognizes an input recognition error, the processor 250 may correct the input recognition error. In this case, the processor 250 may re-request the user to input data or determine the user's intention through a query and then correct the error.

The processor 250 may continuously collect and learn the decision and response of the user according to the operation of the user input recognition device 200 through P300 EEG.

As described above, according to the present disclosure, it is possible to recognize an error through the EEG, thereby correcting the error more quickly than in a case in which a user transfers information about the error to the system in a different method.

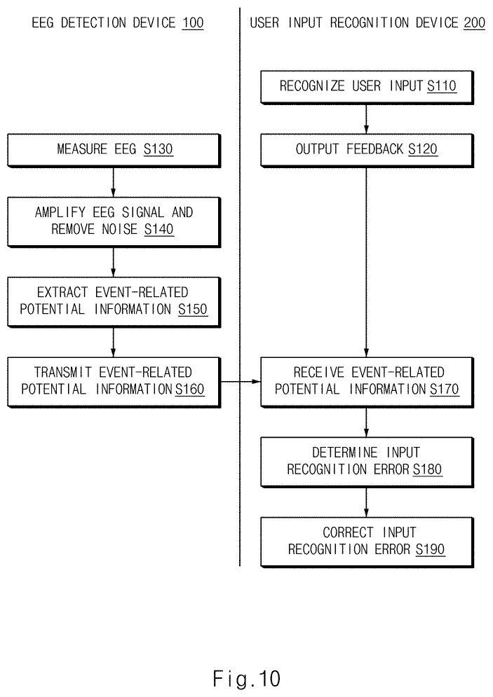

FIG. 10 is a flowchart illustrating a method of operating a user interface system according to an embodiment of the present disclosure.

The user input recognition device 200 may recognize a user's input through the input recognition device 220 (S110). For example, the processor 250 of the user input recognition device 200 may recognize a speech command (speech input) through the input recognition device 220 when the user utters a speech command.

The user input recognition device 200 may output a feedback based on a result of recognizing the user's input (S120). For example, the processor 250 of the user input recognition device 200 may perform an operation according to the speech command recognized through the input recognition device 220 and output a speech guide as the feedback. In this case, the user input recognition device 200 may transmit the feedback information to the EEG detection device 100. The feedback information may include whether the feedback is output and the feedback output time point, and the like.

The EEG detection device 100 may measure the EEG of the user (S130). The EEG detection device 100 may receive an EEG signal through the electrode 110. The electrode 110 may be made of an ultra-thin film of 1pm or less.

The EEG detection device 100 may amplify the EEG signal input through the electrode 110 and remove noise (S140). The first amplifier 120 of the EEG detection device 100 may primarily amplify the EEG signal input through the electrode 110. The noise filter 130 may remove noise included in the EEG signal primarily amplified. In addition, the second amplifier 140 of the EEG detection device 100 may secondly amplify the EEG signal from which the noise is removed and output the EEG signal to the controller 150.

The EEG detection device 100 may extract event-related potential information from the EEG signal (S150). The controller 150 may extract EEG waveforms of up to 500 msec after a feedback outputs from the EEG signal as the event-related potential information. The event-related potential may be a change in a potential occurring in the brain of the user with respect to the feedback output from the user input recognition device 200.

The EEG detection device 100 may transmit the event-related potential information to the user input recognition device 200 (S160). The EEG detection device 100 may transmit event-related potential information to the user input recognition device 200 using wireless communication such as Bluetooth.

The user input recognition device 200 may receive event-related potential information transmitted from the EEG detection device 100 (S170). The user input recognition device 200 may receive event-related potential information in real time along with the EEG detection device 100 using wireless communication such as Bluetooth.

The user input recognition device 200 may analyze event-related potential information to determine whether an input recognition error is present (S180). The user input recognition device 200 may determine whether a P300 potential component exists in the event-related potential information. The user input recognition device 200 may determine that an input recognition error occurs when the P300 potential component in the event-related potential information is found. On the other hand, when the P300 potential component in the event-related potential information is not found, the user input recognition device 200 may determine that the input recognition in which the error does not occur is normal (successful). The P300 potential component may be a peak appearing around 300 msec after output of the feedback.

The user input recognition device 200 may correct an input recognition error (S190). When the user input recognition device 200 recognizes an input recognition error, the user input recognition device 200 may correct the error by requesting of the re-input of user data or determining the intention of the user through a query.

The embodiments of the present disclosure are provided to explain the spirit and scope of the present disclosure, not to limit them, so that the spirit and scope of the present disclosure is not limited by the embodiments. The scope of the present disclosure should be construed on the basis of the accompanying claims, and all the technical ideas within the scope equivalent to the claims should be included in the scope of the present disclosure.

According to the present disclosure, it is possible to improve speech recognition accuracy by analyzing an event-related potential pattern through measurement of the user's EEG and recognize and correct a user input recognition error based on the analysis result.

Hereinabove, although the present disclosure has been described with reference to embodiments and the accompanying drawings, the present disclosure is not limited thereto. The embodiments and the disclosure may be variously modified and altered by those having ordinary skill in the art to which the present disclosure pertains without departing from the spirit and scope of the present disclosure claimed in the following claims.

What is claimed is:

1. A user interface system comprising:
an electroencephalogram (EEG) detection device configured to detect event-related potential information by measuring an EEG of a user; and
a user input recognition device configured to recognize and correct an input recognition error by analyzing the event-related potential information when an input of the user is recognized.

2. The user interface system of claim 1, wherein the event-related potential is a potential change occurring in a brain of the user with respect to a feedback output from the user input recognition device.

3. The user interface system of claim 1, wherein the EEG detection device includes
an electrode attached to a scalp of the user to receive an EEG signal,
a first amplifier configured to primarily amplify the EEG signal,
a noise filter configured to remove noise from the EEG signal primarily amplified by the first amplifier,
a second amplifier configured to secondly amplify the EEG signal from which the noise has been removed,
a controller configured to extract the event-related potential information from the EEG signal secondly amplified by the second amplifier, and
a first communication device configured to transmit the event-related potential information to the user input recognition device using wireless communication according to an instruction of the controller.

4. The user interface system of claim 3, wherein the electrode is made of an ultra-thin film of 1 μm or less.

5. The user interface system of claim 4, wherein the noise filter is implemented with a Driven Right Leg Circuit (DRLC) including two OP-AMPs and resistors.

6. The user interface system of claim 1, wherein the user input recognition device includes
a second communication unit configured to receive the event-related potential information transmitted from the EEG detection device through wireless communication,
an input recognition device configured to recognize the input of the user, and
a processor configured to output a feedback according to a recognition result of the input of the user of the input recognition device and recognize and correct an input recognition error based on the event-related potential information.

7. The user interface system of claim 6, wherein the processor determines the input recognition error when a P300 potential component in the event-related potential information is found.

8. The user interface system of claim 7, wherein the P300 potential component is a peak appearing around 300 msec after output of the feedback.

9. The user interface system of claim 6, wherein the processor corrects the input recognition error by requesting the user to re-input data or identifying an intention of the user through a query.

10. A method of operating a user interface system, comprising:
detecting event-related potential information through measurement of an electroencephalogram (EEG) of a user when recognizing an input of the user,
recognizing an input recognition error by analyzing the event-related potential information, and
correcting the input recognition error.

11. The method of claim 10, wherein the event-related potential is a potential change occurring in a brain of the user with respect to a feedback output from a user input recognition device.

12. The method of claim 10, wherein the detecting of the event-related potential information includes
recognizing the input of the user,
outputting a feedback according to a result of recognizing the input,
measuring a EEG signal of the user upon output of the feedback, and
extracting the event-related potential information from the EEG signal.

13. The method of claim 12, wherein the measuring of the EEG signal of the user includes
primarily amplifying the EEG signal,
removing noise included in the primarily-amplified EEG signal, and
secondly amplifying the EEG signal from which the noise has been removed.

14. The method of claim 10, wherein the recognizing of the input recognition error includes determining that an input recognition error occurs when a P300 potential component is found in the event-related potential information.

15. The method of claim 14, wherein the P300 potential component is a peak appearing around 300 msec after output of the feedback.

16. The method of claim 10, wherein the correcting of the input recognition error includes correcting the input recognition error by requesting the user to re-input data or identifying an intention of the user through a query.
